(19) United States
(12) Patent Application Publication
(10) Pub. No.: US 2008/0097389 A1
(43) Pub. Date: Apr. 24, 2008

(54) Y-type blood transfusion set

(76) Inventor: Roland B. Wilson, Wabash, IN (US)

Correspondence Address: BUTZEL LONG, 350 SOUTH MAIN STREET, SUITE 300, ANN ARBOR, MI, 48104, US

(21) Appl. No.: 11/483,793

(22) Filed: Jul. 10, 2006

(51) Int. Cl. A61M 25/14

(52) U.S. Cl. 604/518; 604/264; 604/82; 604/284

(57) ABSTRACT

A-type blood transfusion set having a blood limb, a saline limp and common limb and a one-way saline valve provided in the saline limb which only allows fluid within the saline limb to flow toward the Y connector. The blood limb and saline limb are color coded or marked with some type of identification so that, prior to coupling the blood limb to a source of blood or the saline limb to a source of saline, visual identification of the saline limb, having the one-way saline valve therein, can be readily achieved.

### TECHNICAL FIELD

The present invention relates to methods and apparatus for transfusing blood, including packed red blood cells, into a patient which reduce the risk of human error during the transfusion process.

### BACKGROUND ART

A typical present day y-type blood transfusion set has at its proximal aspect two medical tubing segments with their respective complement of attached subcomponents. These upper limb assemblies are identical to each other in every aspect and minimally include the following basic features: (1) spikes are provided at the uppermost ends of the upper limb assemblies, (2) distal ends of the upper limb assemblies connect to each other (converge) at an open y-intersect, (3) the lengths of each of the upper limbs are the same (spike-to-y-intersect), and (4) each of the upper limbs has only one external clamp which is coaxially maneuverable up and down. The external clamp can be manually adjusted to vary the rate of flow by gradually diminishing a short segment of the enclosed tubular space (intraluminal cross-sectional area) from full patency to the point of complete occlusion, and vice versa.

Since these upper limb assemblies are identical in appearance (color, size, shape, etc.), weight, biomedical composition, and so on, the blood transfusionist can equally select either limb to spike a bag of saline or a bag of blood. However, depending on the contents of the container that is spiked, that particular limb assembly will thereafter be accordingly designated the saline limb or the blood limb.

The next important subcomponent of a typical present day y-type blood transfusion set below the y-intersect is the blood filter. Next is a medical tubing segment with its own complement of attached subcomponents. This “common limb” is always longer than the saline and blood limbs and minimally has a manually-controlled, coaxially maneuverable external clamp (just like the saline and blood limbs) and usually one side port. The distal end of the common limb connects intravenously to the patient either (1) directly via an angioaccess device, such as the hub of an intravenous catheter or (2) first via a blood-warming apparatus, which in turn connects to an angioaccess device.

Otherwise, different brands of present-day y-blood transfusion sets may differ amongst each other in less important (or secondary) respects that do not alter their basic function, such as, for example, variability in lengths of the limbs, different types of subcomponents, number and location of tubing side ports, one or two additional external clamps on the common limb, etc. (The tubing side ports typically provide access for a hypodermic needle or a needless system.)

Typically, the transfusionist sequentially proceeds through the following initial steps in preparation to transfuse a patient with blood using a present-day y-type blood transfusion set:

1. All three external clamps (saline, blood and common limbs) are closed.

2. A saline bag is spiked (this generic limb is thereafter functionally designated as the saline limb)

3. The y-set is primed by opening the saline and common limb clamps thereby allowing the saline to stream all the way to the distal end of the common limb, or to the distal end of the attached blood warming device (a blood transfusion y-set should never be primed with a blood unit).

4. Any potentially threatening air bubbles or columns of air are removed from the system.

5. The saline flow is stopped by manipulating the saline and/or common limb clamp(s).

6. The distal end of the y-set or the distal end of the blood-warming apparatus is connected to the patient via an angioaccess device.

7. The clamp(s) that were closed in step 5 (both the saline and common limb clamps) are open thereby allowing the saline to flow into the patient in order to verify the readiness/patency of the system (the blood limb clamp remain closed).

In the United States at least eighty percent of blood transfusions occur in hospital emergency departments or operating rooms. The most common blood product that is transfused is packed red blood cells which are prepared by removing most of the plasma and coagulation factors from donated whole blood, leaving mostly concentrated red blood cells. Whole blood transfusions are generally rare, because donated whole blood almost always has its components (red blood cells, platelets, coagulation factors, etc.) removed separately to specially-marked containers for specific transfusion needs.

There exists two dominate schools of thought on how to transfuse packed whole red blood cells after step 7 above.

School A

School A prefers to reconstitute (dilute) the bag of packed red blood cells with the adjacent bag of saline prior to transfusion and follows the next steps (after steps 1-7 above):

8. The common limb clamp is closed (the saline limb clamp is left open).

9. The bag of packed red blood cells is spiked with the remaining spike (the blood limb clamp is still closed from step 1).

10. The saline bag is hung higher that the blood bag and/or manually squeezed (or mechanically compressed).

11. The blood limb clamp is opened.

12. The rapid flow of saline down the saline limb and then up to the blood limb from the y-intersect junction is monitored (normally reconstitution of the packed red blood cells is complete in less than one minute).

13. A determination is made as to if the bag of red blood cells has been adequately reconstituted.

14. The saline clamp is closed (the blood clamp is left open).

15. The common limb clamp is opened.

16. A visual determination is made that the blood is truly flowing to the patient.

School B

School B prefers to transfuse the bag of packed red blood cells directly after spiking (no prior reconstitution) and follows the next steps (after steps 1-7 above):

8. The saline limb is clamp is closed.

9. The bag of packed red blood cells is spiked with the remaining spike (blood limb clamp is still closed from step 1).

10. The blood limb clamp is opened.

11. A visual determination is made that the blood is truly flowing to the patient.

In either of the aforementioned transfusion scenarios, if the situation is really urgent, the transfusionist often inserts the bag of blood into a pneumatic compression device, which he or she quickly inflates up to 300 mm of mercury or higher in a matter of seconds. Under these conditions the bag of blood will empty in a matter of minutes.

There are a number of potential problems associated with the above transfusion methods. A primary human error occurs when there is a failure to close the saline limb clap prior to actual blood transfusion (step 14 in School A and step 8 in School B). As a consequence, the blood product that is intended to infuse into the patient will, instead, flow down the blood limb and then up the saline limb from the y-intersect and into the saline bag. This occurs even if the common limb clamp is fully open because the cumulative resistance factors that are downstream from the y-intersect (e.g., primarily the blood filter and the length and radius of the common limb) are significantly higher than the gravitational force to pull the blood down to the patient. The loss of blood into the saline bag can be especially rapid if the blood bag is pressurized s noted above. Although the blood can still be transfused to the patient, valuable time can be lost because resuscitation of the blood product is delayed. How much time can pass before such an error is realized depends on the situation. This error (bypassed process step) can occur by the physician and/or hospital personal in the chaotic scenario of trying to emergently resuscitate a patient who just lost, or is acutely losing, a massive amount of blood. In fact, the human error is most likely to occur while the transfusionist is tying to accomplish several tasks simultaneously, such as applying monitors, trying to identify the source(s) of bleeding, physically assessing and reassessing the patient, trying to get a history (more difficult if the patient is semiconscious or unconscious), securing an airway on a vomiting patient, starting another intravenous line, coordinating other healthcare personnel, and so on.

To compound the problem, the transfusionist may divert his or her attention prematurely to another task (hurriedly thinking ahead before completing the task at hand) before verifying true blood flow to the patient in occurring (step 16 in School A and step 11 in School B). This second human error occurs when the transfusionist does not promptly realize the mistake above (this error is more likely to be committed by a relatively inexperienced or situationally distracted transfusionist or his or her designee). What transpires is that the transfusionist incorrectly assumes that true blood transfusion has begun by visualizing (1) the degree of emptying of the blood bag and/or (2) blood streaming down the blood limb, and not noticing that (3) blood is not streaming downward beyond the y-intersect, and/or (4) blood is running up the saline limb. On the contrary, true blood flow to the patient truly begins only when blood is flowing downward past the y-intersect, and the saline limb and bag simultaneously retain their colorless appearance. These four confirmation steps can be accomplished in less than ten seconds.

The present invention provides a solution to preventing the type or human error noted above which can lead to disastrous consequences and medical liabilities.

### DISCLOSURE OF THE INVENTION

According to various features, characteristics and embodiments of the present invention which will become apparent as the description thereof proceeds, the present invention provides a y-type blood transfusion set having a blood limb, saline limp and common limb which are coupled together at a Y connector, the improvement comprising a one-way saline valve provided in the saline limb which only allows fluid within the saline limb to flow toward the Y connector.

In a further embodiment, one of a distinguishing color or indicia associated with at least one of the blood limb or saline limb is provided in for to visually differentiate the blood limb from the saline limb.

The present invention further provides a y-type blood transfusion set which comprises:

a blood limb that includes:
a length of tubing having first and second ends,
a spike coupled to the first end of the tubing, and
an external flow regulator provided on the tubing;

a saline limb that includes:
a length of tubing having first and second ends,
a spike coupled to the first end of the tubing,
an external flow regulator provided on the tubing, and
a one-way saline valve provided between the first end of the tubing and the external flow regulator;

a common limb having a first and second ends; and

a Y-connector coupled to the second ends of each of the lengths of tubing of the blood limb and the saline limb and the first end of the common limb.

The present invention further provides a method of transfusing blood into a patient using a y-type blood transfusion set having a blood limb, saline limp and common limb which are coupled together at a Y connector, the method comprising the steps of:

providing a one-way saline valve provided in the saline limb which only allows fluid within the saline limb to flow toward the Y connector;

coupling the blood limb to source of a blood product;

coupling the saline limb to a source of saline;

coupling the common limb to a patient; and

allowing the one-way saline valve to prevent fluid flow towards the source of saline while transfusing blood into the patient.

### BRIEF DESCRIPTION OF DRAWINGS

The present invention will be described with reference to the attached drawings which are given as non-limiting examples only, in which:

FIG. 1 is a schematic diagram of a prior art y-type blood transfusion set.

FIG. 2 is a schematic diagram of a y-type blood transfusion set according to one embodiment of the present invention.

### BEST MODE FOR CARRYING OUT THE INVENTION

The present invention is directed to methods and apparatus for transfusing blood, including packed red blood cells, into patients which methods and apparatus reduce the risk of human error during transfusion procedures. The present invention provides a y-type blood transfusion set that includes a blood limb, a saline limb and a common limb that are coupled together at a common y-intersect and a means for preventing the backflow of fluid through at least a portion of the saline limb. In addition, the present invention provides a system for identifying and distinguishing the blood limb from the saline limb.

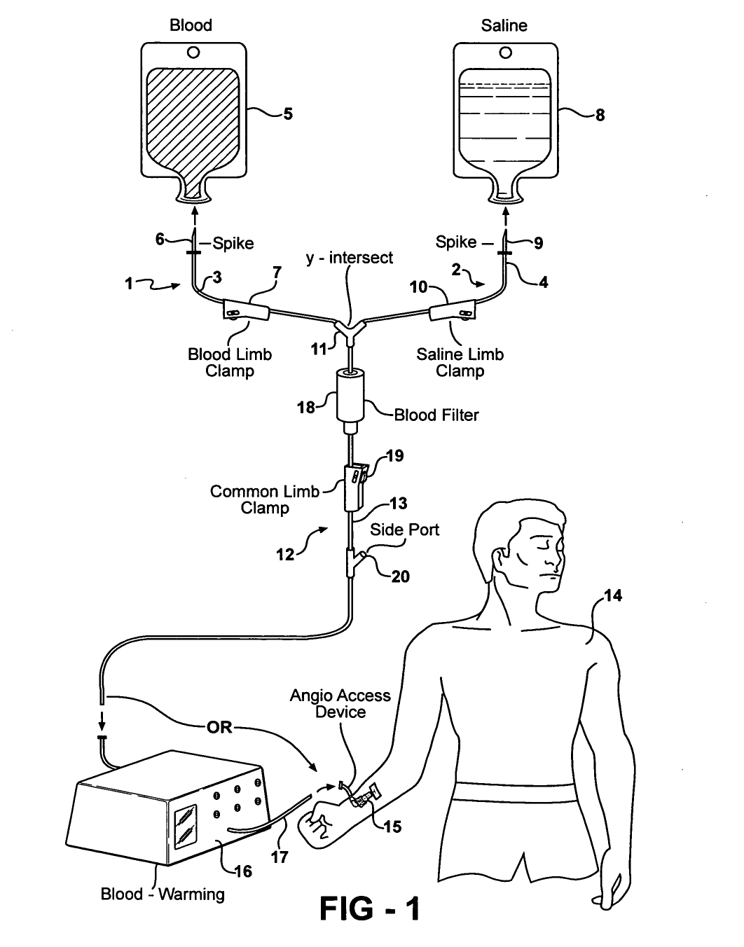

FIG. 1 is a schematic diagram of a prior art y-type blood transfusion set. As shown, the y-type blood transfusion set includes two upper branches or limbs which are commonly referred to as the blood limb 1 and the saline limb 2. The two upper branches or limbs comprise lengths of clear tubing 3 and 4 which typically have substantially equal lengths. The upper limb designated as the blood limb 1 is configured to be fluidly coupled to a bag 5 of packed red blood cells which are reconstituted or diluted during a blood transfusion process as discussed above. The upper end of the tubing 3 which corresponds to the blood limb 1 is coupled to a bag coupling device that is commonly referred to as a spike 6. The spike 6 is configured to be coupled to the bag 5 containing the packed red blood cells as, or after, the spike 6 penetrates the bag 5, thereby establishing fluid communication between the tubing 3 the corresponds to the blood limb 1 and the contents of the bag 5.

The blood limb 1 is provided with an external flow regulator 7 which can be of conventional design, such as a roll clamp, pinch clamp, etc. Manually operating or adjusting the external flow regulator 7 provides control of fluid flow within the blood limb 1.

The upper limb designated as the saline limb 2 is configured to be fluidly coupled to a bag 8 of saline which is used to reconstitute or dilute the packed blood cells in bag 1 during a blood transfusion process as discussed above. The upper end of the tubing 4 which corresponds to the saline limb 2 is also coupled to a bag coupling device that is commonly referred to as a spike 9. The spike 9 is configured to be coupled to the bag 8 containing the saline as, or after, the spike 9 penetrates the bag 8, thereby establishing fluid communication between the tubing 4 that corresponds to the saline limb 2 and the contents of the bag 8.

The saline limb 2 is provided with an external flow regulator 10 which can be of conventional design, such as a roll clamp, pinch clamp, etc. Manually operating or adjusting the external flow regulator 10 provides control of the fluid flow within the saline limb 2.

The lengths of tubing 3 and 4 which correspond to the blood limb 1 and the saline limb 2 merge and are fluidly coupled together at the y-intersect 11 which can be a conventional Y connector. A lower limb, typically referred to as the common limb 12, is coupled to and extends from the y-intersect 11. The common limb 12 comprises a length of clear tubing 13 which has a distal end (distal from the y-intersect) that is eventually coupled intravenously to the patient 14 via either an angioaccess device 15, such as the hub of an intravenous catheter, or is coupled to a blood-warming apparatus 16, which in turn connects to an angioaccess device 15.

In the prior art y-type blood transfusion set shown in FIG. 1, the distal end of the tubing 13 which corresponds to the common limb 12 is coupled to a blood warming device 16 which can be of conventional design. The blood warming device 16 is connected by a length of tubing 17 to an angioaccess device that is generally identified by reference numeral 15 which can be a conventional device, such as the hub of an intravenous catheter.

The common limb 12 of the y-type blood transfusion set shown in FIG. 1 is provided with a blood filter 18 which can be or conventional designed and used, for example, to remove solid microemboli from the reconstituted red blood cells. The common limb 12 is provided with an external flow regulator 19 downstream of the blood filter. The external flow regulator 19 can be of conventional design, such as a roll clamp, pinch clamp, etc. Manually operating or adjusting the external flow regulator 19 provides for control of fluid flow within the common limb 12.

The common limb 12 shown in FIG. 1 is provided with an injection portal 20, which allows for the injection of various fluids and medications into the common limb 12. In addition, additional intravascular tubes, from other sources, can be inserted into the injection portal 20 in a “piggyback” fashion.

As noted above, and shown in FIG. 1, the upper branches or limbs (blood limb 1 and saline limb 2) of the prior art y-type intersect blood transfusion set are identical to each other in every aspect so that their appearance is substantially identical. This similarity and identical appearance attributes to the human errors discussed above which can have adverse if not disastrous consequences.

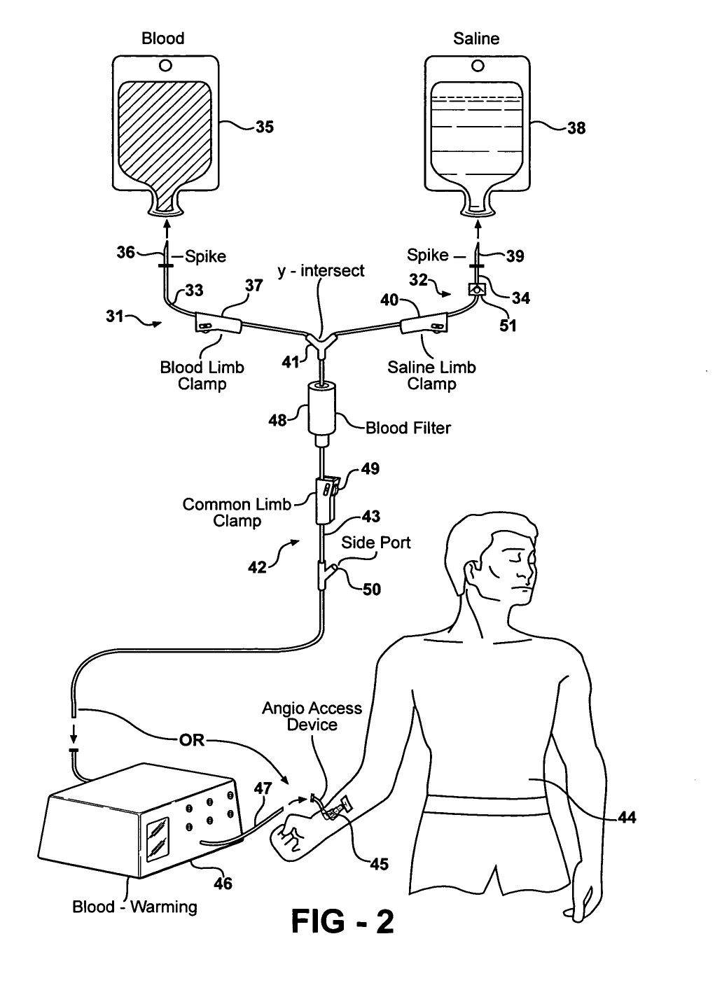

FIG. 2 is a schematic diagram of a y-type blood transfusion set according to one embodiment of the present invention. The y-type blood transfusion set depicted in FIG. 2 comprises an upper blood limb 31 and a lower common limb 42 which, as depicted, can include substantially the same elements or components of the blood limb 1 and common limb 12 of the y-intersect blood transfusion set shown in FIG. 1 which can further be configured in substantially the same manner.

The blood limb 31 is configured to be fluidly coupled to a bag 35 of packed red blood cells which are reconstituted or diluted during a blood transfusion process as discussed above. The upper end of the tubing 33 which corresponds to the blood limb 31 is coupled to a bag coupling device that is commonly referred to as a spike 36. The spike 36 is configured to be coupled to the bag 35 containing the packed red blood cells as, or after, the spike 36 penetrates the bag 35, thereby establishing fluid communication between the tubing 33 the corresponds to the blood limb 31 and the contents of the bag 35.

The blood limb 31 is provided with an external flow regulator 37 which can be of conventional design, such as a roll clamp, pinch clamp, etc. Manually operating or adjusting the external flow regulator 37 provides for control of fluid flow within the blood limb 31.

The lower or common limb 42 comprises a length of clear tubing 43 which has a proximal end that is coupled to the upper limbs (blood limb 31 and saline limb 32) via a y-intersect 41 which can be a conventional Y connector. The distal end (distal from the y-intersect) of the common limb 42 is eventually coupled intravenously to the patient 44 via either an angioaccess device 45, such as the hub of an intravenous catheter, or is coupled to a blood-warming apparatus 46, which in turn connects to an angioaccess device 45.

In the y-type blood transfusion set shown in FIG. 2, the distal end of the tubing 43 which corresponds to the common limb 42 is coupled to a blood warming device 46 which can be of conventional design. The blood warming device 46 is connected by a length of tubing 47 to an angioaccess device that is generally identified by reference numeral 45 which can be a conventional device, such as the hub of an intravenous catheter.

The common limb 42 of the y-type blood transfusion set shown in FIG. 2 is provided with a blood filter 48 which can be or conventional designed and used, for example, to remove solid microemboli from the reconstituted red blood cells. The common limb 42 is also provided with an external flow regulator 49 downstream of the blood filter. The external flow regulator 49 can be of conventional design, such as a roll clamp, pinch clamp, etc. Manually operating or adjusting the external flow regulator 49 provides control off fluid flow within the common limb 42.

The common limb 42 shown in FIG. 2 can be provided with one or more injection portals 50 (one shown), which allow the injection of various fluids and medications into the common limb 42. In addition, additional intravascular tubes, from other sources, can be inserted into the injection portal(s) in a “piggyback” fashion if desired.

The y-type blood transfusion set shown in FIG. 2 also includes an upper saline limb 32 formed from a length of clear tubing 34 that is configured to be fluidly coupled to a bag 38 of saline which is used to reconstitute or dilute the packed blood cells in bag 35 during a blood transfusion process as discussed above. The upper end of the tubing 33 which corresponds to the saline limb 32 is also coupled to a bag coupling device that is commonly referred to as a spike 39. The spike 39 is configured to be coupled to the bag 38 containing the saline as, or after, the spike 39 pierces the bag 38, thereby establishing fluid communication between the tubing 33 that corresponds to the saline limb 31 and the contents of the bag 38.

The saline limb 31 is provided with an external flow regulator 40 which can be of conventional design, such as a roll clamp, pinch clamp, etc. Manually operating or adjusting the external flow regulator 40 provides for control of fluid flow within the saline limb 31.

The saline limb 31 of the y-type blood transfusion set of the present invention includes a one-way saline valve 51 between the spike 39 and external flow regulator 40. The one-way saline valve 51 can be of any type of conventional design associated with one-way fluid valves and, during use of the y-type blood transfusion set in a blood transfusion procedure, will prevent any fluid within the saline limb 31 from flowing towards the bag 38 containing the saline. Moreover, the one-way saline valve 51 will prevent any fluid within either the blood limb 31 or common limb 42 from flowing into the saline limb 32; howbeit, fluid or fluid components within either the blood limb 31 or common limb 42 can diffuse into the lower portion of the saline limb 32, i.e. downstream of the one-way saline valve 51.

The inclusion of the saline one-way valve 51 in the saline limb 32 presents a fail-safe provision which will compensate should the primary human error noted above occur. That is, if there is a failure to close the saline limb clap 40 during use and prior to actual blood transfusion (step 14 in School A and step 8 in School B above), the one-way saline valve 51 prevents the blood product that is intended to infuse into the patient from flowing down the blood limb 31 and then up the saline limb 32 from the y-intersect 41 and into the saline bag 38.

The one-way saline valve 51 can be incorporated into the y-type blood transfusion set, i.e. into the saline limb 32, when the y-type blood transfusion set is manufactured, or incorporated into the saline limb 32 on site.

The tradeoff of incorporating the one-way saline valve 51 into the y-type blood transfusion set is that the transfusionist must remember to spike the saline bag 38 with the length of tubing 34 that includes the one-way saline valve 51. The presence of the one-way saline valve 51 itself provides a means by which the transfusionist can readily identify the saline limb 32 from the blood limb 31.

Although the presence of the one-way saline valve 51 itself provides a means for visually identifying the saline limb 32 from the blood limb 31, the present invention further provides alternative embodiments in which the saline limb 32 and/or blood limb 31 of a y-type blood transfusion set is manufactured or constructed in such a way so that these limbs can be readily differentiated from one another.

According to one embodiment, at least one of the saline limb 32 and the blood limb 32 are color coded in some manner. For example, the saline limb 32 could be color coded in blue to symbolize water or/and the blood limb 31 could be color coded in red to symbolize blood. Such color coding could involve coloring or tinting any one of the respective spike 39 or 36 and/or tubing 34 or 33 of the saline and/or blood limb 32, 31 or providing auxiliary color coded tags attached to the respective saline and/or blood limbs 32, 31. The tubing could be completely colored or tinted or could be partially colored or tinted, e.g., have colored or tinted strips or some other pattern of colored indicia, including symbols, text, numbers, etc. thereon or incorporated therein. In an alternative to color coding, suitable tags or markers could be provided on the saline and/or blood limb 32, 31 to help identify the limbs. For example, clip-on or snap-on identification elements similar to those disclosed in U.S. Application Publication No. 2004/0243054 to Kraushaar (the entire disclosure of which in hereby expressly incorporated herein by reference) could be provided and configured to connect to the tubing 34 or 33 and/or external flow regulator 40 or 37 of one of both of the saline limb and/or blood limbs 32, 31. In such an instance, the identification elements could include indicia such as symbols, text, numbers, etc. and/or be color coded. Removable or non-removable identification elements, tags or markers could also be used, including removable caps or covers for the spikes 39 or 36 that are color coded or labeled with indicia. It is also possible to provide indicia such as symbols, text, numbers, etc. on the tubing, spikes 39, 36 and/or external flow regulators 40, 37, such as for example the words “SALINE” and “BLOOD” in any color. Essentially any means for providing visual differentiation for the saline limb 32 and the blood limb 31 can be used in conjunction with a one-way saline valve 51 according to the present invention.

Although the present invention has been described with reference to particular means, materials and embodiments, from the foregoing description, one skilled in the art can easily ascertain the essential characteristics of the present invention and various changes and modifications can be made to adapt the various uses and characteristics without departing from the spirit and scope of the present invention as described above.

What is claimed is:

1. In a y-type blood transfusion set having a blood limb, saline limp and common limb which are coupled together at a Y connector, the improvement comprising a one-way saline valve provided in the saline limb which only allows fluid within the saline limb to flow toward the Y connector.

2. A y-type blood transfusion set according to claim 1, further comprising at least one of a distinguishing color or indicia associated with at least one of the blood limb or saline limb which provides for visually differentiating the blood limb from the saline limb.

3. A y-type blood transfusion set according to claim 2, wherein at least one of the blood limb and the saline limb are at least partially color coded with a distinguishing color.

4. A y-type blood transfusion set according to claim 3, wherein each of the blood limb and the saline limb are at least partially color coded with a different color.

5. A y-type blood transfusion set according to claim 2, wherein at least one of the blood limb and the saline limb are provided with differentiating indicia.

6. A y-type blood transfusion set according to claim 5, wherein the differentiating indicia comprises at least one of symbols, text and numbers.

7. A y-type blood transfusion set according to claim 2, wherein each of the blood limb and saline limb includes a spike for fluidly coupling respective limb to a bag or bottle of fluid and the spike or at least one of the blood limb or saline limb is provided with the distinguishing color or indicia.

8. A y-type blood transfusion set which comprises:
a blood limb that includes:
a length of tubing having first and second ends,
a spike coupled to the first end of the tubing, and
an external flow regulator provided on the tubing;
a saline limb that includes:
a length of tubing having first and second ends,
a spike coupled to the first end of the tubing,
an external flow regulator provided on the tubing, and
a one-way saline valve provided between the first end of the tubing and the external flow regulator;
a common limb having a first and second ends; and
a Y-connector coupled to the second ends of each of the lengths of tubing of the blood limb and the saline limb and the first end of the common limb.

9. A y-type blood transfusion set according to claim 8, further comprising at least one of a distinguishing color or indicia associated with at least one of the blood limb or saline limb which provides for visually differentiating the blood limb from the saline limb.

10. A y-type blood transfusion set according to claim 9, wherein at least one of the blood limb and the saline limb are at least partially color coded with a distinguishing color.

11. A y-type blood transfusion set according to claim 10, wherein each of the blood limb and the saline limb are at least partially color coded with a different color.

12. A y-type blood transfusion set according to claim 8, wherein at least one of the blood limb and the saline limb are provided with differentiating indicia.

13. A y-type blood transfusion set according to claim 12, wherein the differentiating indicia comprises at least one of symbols, text and numbers.

14. A y-type blood transfusion set according to claim 8, wherein each of the blood limb and saline limb includes a spike for fluidly coupling respective limb to a bag or bottle of fluid and the spike or at least one of the blood limb or saline limb is provided with the distinguishing color or indicia.

15. A y-type blood transfusion set according to claim 8, wherein the common limb further comprises an external flow regulator on the length of tubing of the common limb.

16. A y-type blood transfusion set according to claim 15, wherein the common limb further comprises at least one injection portal in fluid communication to the length of tubing of the common limb.

17. A y-type blood transfusion set according to claim 15, wherein the common limb further comprises a blood filter.

18. A method of transfusing blood into a patient using a y-type blood transfusion set having a blood limb, saline limp and common limb which are coupled together at a Y connector, the method comprising the steps of:
providing a one-way saline valve provided in the saline limb which only allows fluid within the saline limb to flow toward the Y connector;
coupling the blood limb to source of a blood product;
coupling the saline limb to a source of saline;
coupling the common limb to a patient; and
allowing the one-way saline valve to prevent fluid flow towards the source of saline while transfusing blood into the patient.

19. A method of transfusing blood into a patient according to claim 18, further comprising warming the blood before the blood is transfused into the patient.

20. A method of transfusing blood into a patient, further comprising filtering the blood as it flows through the common limb.
